(19) United States
(12) Patent Application Publication
(10) Pub. No.: US 2026/0226373 A1
(43) Pub. Date: Aug. 6, 2026

(54) DETERGENT HAVING INCREASED WASHING POWER AGAINST OILY AND GREASY SOILING

(71) Applicant: HENKEL AG & CO. KGAA, Düsseldorf (DE)

(72) Inventors: Mahdis HESAMI, Düsseldorf (DE); Peter SCHMIEDEL, Düsseldorf (DE); Anja VON KATHEN, Wuppertal (DE)

(21) Appl. No.: 19/146,294

(22) PCT Filed: Dec. 8, 2023
(86) PCT No.: PCT/EP2023/084952

§ 371 Date: Jul. 8, 2025

(30) Foreign Application Priority Data
Jan. 17, 2023 (DE) 10 2023 200 330.9

(51) Int. Cl. C11D 3/37; C08G 83/00; C11D 17/04

(52) U.S. Cl. CPC C11D 3/3715; C08G 83/005; C11D 17/043

(57) ABSTRACT

Particular compounds increase the washing power of detergents against oily and greasy soiling using compounds of general formula (I) in which each R is —H and —O—C(═O)—R′, and R′ is selected from alkyl groups having 6 to 10 C atoms and mixtures thereof, and in which at least 5% of the number of functional groups R are those which are not H.

The present invention relates to textile detergents containing a specific star polymer, to a textile washing method using such a star polymer, and to the use of the star polymer to increase the washing performance of textile detergents against grease- or oil-based soiling.

As a contribution to combating climate change, one of the most important goals in textile washing is to reduce CO₂ emissions per wash cycle. This can be achieved by lowering the washing temperature. In order to ensure that detergents used in textile washing have the desired washing power even under cold washing conditions, so-called washing power enhancers are often used.

Another important aim is the use of biodegradable detergent ingredients in order to avoid the accumulation of substances that either do not degrade or do not easily degrade in the ecosystem wherever possible.

The object of the present invention is to achieve both aims simultaneously. An element that also stands in the way of achieving the aims is that when the washing temperature is reduced below the melting temperature range of oily soiling, the soiling hardens into greasy soiling and thus becomes even more difficult to remove.

International patent application WO 2022/136389 A1 proposes the use of certain alkoxylated polyamines to improve washing properties under cold washing conditions, and international patent application WO 2017/011733 A1 discloses certain cyclic amines as active ingredients that can be used under cold washing conditions to increase washing power. International patent application WO 2014/095540 A1 teaches that alkoxylated polyamines have a particularly good washing power-enhancing effect when combined with certain nonionic surfactants.

Surprisingly, it has been found that polymers with completely different structures increase washing power under cold washing conditions.

The invention relates to the use of a compound of general formula (I),

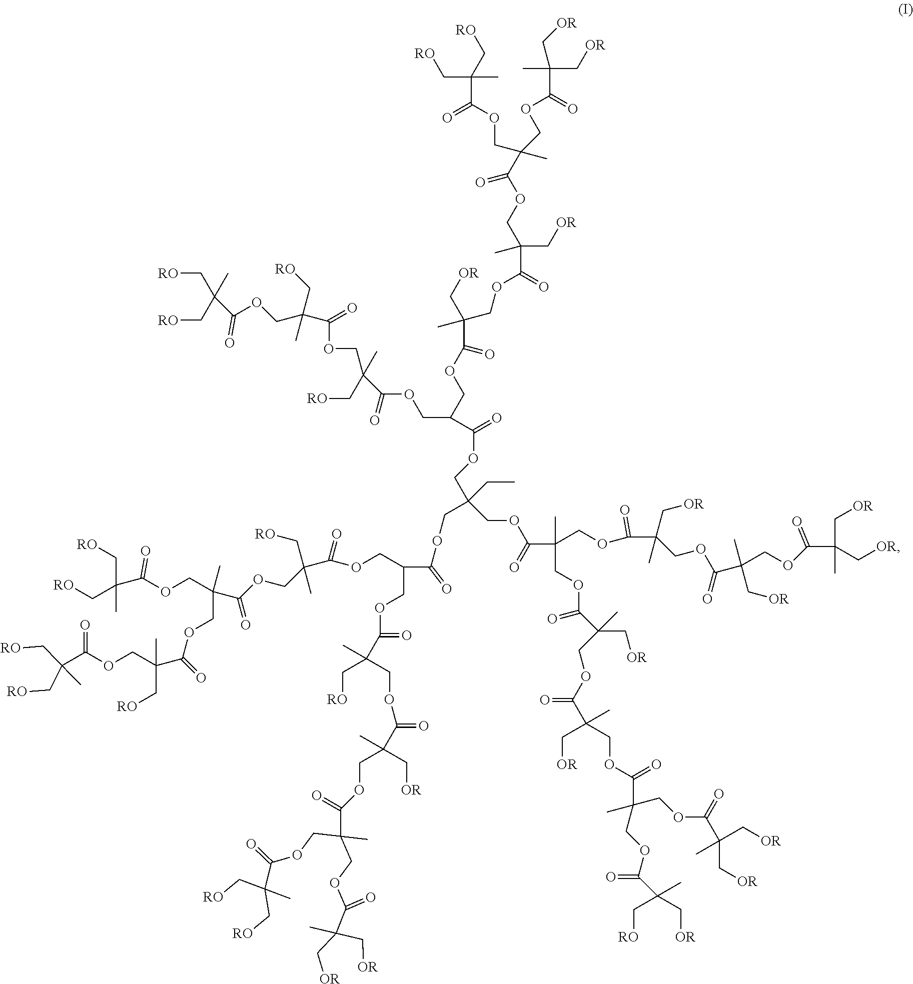

in which each R is selected from —H and —O—C(═O)—R′, and R′ is selected from the alkyl groups having 6 to 10 C atoms and mixtures thereof, wherein at least 5% of the number of functional groups R are those which are not H,
to increase the washing power of detergents against grease- or oil-based textile soiling.

In the compound of general formula (I), 20% to 35%, in particular 25% to 30% of the number of functional groups R are preferably not H. The functional groups R′ are preferably linear but can also be branched; they are preferably part of a caproic acid, enanthic acid, caprylic acid, pelargonic acid or capric acid functional group-O—C(═O)—R′, wherein not all functional groups R′ have to be the same.

Further preferably, in the compound of general formula (I), 35% to 65%, in particular 40% to 60% of the number of functional groups R′ in the functional groups R which are not H are those having a C₇ alkyl group, and 65% to 35%, in particular 60% to 40% of the number of functional groups R′ in the functional groups R which are not H are those having a C₉ alkyl group.

The compounds of general formula (I) are biodegradable. They are obtainable by esterification of 1 molar equivalent of 2-ethyl-2-(hydroxymethyl)-propane-1,3-diol (also called trimethylolpropane) with 29 molar equivalents of 2,2-bis(hydroxymethyl)-propionic acid (also called dimethylolpropionic acid) and subsequent esterification with carboxylic acids R′—COOH, wherein so many molar equivalents of the latter are used that the above-mentioned degrees of esterification of at least 5%, in particular 20% to 35% and particularly preferably 25% to 30% are achieved.

The invention further relates to a method for removing grease- or oil-based soiling from textiles, characterized in that textiles requiring cleaning of this kind are brought into contact with water and a compound of general formula (I) or a detergent and a compound of general formula (I). This method can be carried out manually or mechanically, for example using a domestic washing machine. It is possible to use the detergent and the compound of general formula (I) simultaneously or sequentially. The two can be used simultaneously in a particularly advantageous manner by using an agent, in particular a liquid agent, which contains the compound of general formula (I). The concentration of the compound of general formula (I) in the aqueous washing liquor is preferably 0.03 mg/l to 1 mg/l, in particular 0.05 mg/l to 0.09 mg/l. It is particularly advantageous that the method can be carried out at temperatures of from room temperature to 60° C., in particular in the range of from 20° C. to 40° C.; preferred embodiments of the method according to the invention work with this range. Preferably, the compound of general formula (I) remains in contact with the textile for a period of time in the range from 80 minutes to 160 minutes, in particular from 90 minutes to 110 minutes; the textile is then rinsed and dried in the usual manner.

The use according to the invention in detergents is preferably carried out by using the compound of general formula (I) in an amount of 1 wt. % to 6 wt. %, in particular 2 wt. % to 4 wt. %, wherein “wt. %” statements here and in the following relate to the weight of the total detergent, unless otherwise stated. The invention therefore also relates to a detergent containing a compound of general formula (I), in particular in the amounts indicated in the preceding sentence.

Detergents which contain the compound to be used according to the invention or are used together therewith or are used in the method according to the invention may contain all other conventional constituents of such detergents that do not undesirably interact with an active ingredient that is essential to the invention. Preferably, the detergent is liquid.

Surprisingly, it has been found that the compounds of general formula (I) have a positive influence on the effect of certain other detergent and cleaning agent ingredients and that, conversely, the effect of the compounds of general formula (I) is further enhanced by certain other ingredients. It is therefore preferred that the detergent contains 10 wt. % to 40 wt. %, in particular 20 wt. % to 30 wt. % synthetic anionic surfactant, in particular of the sulfate or sulfonate type, and/or 20 wt. % to 40 wt. %, in particular 25 wt. % to 35 wt. % nonionic surfactant and/or 5 wt. % to 20 wt. %, in particular 7 wt. % to 15 wt. % soap.

Synthetic anionic surfactants which are particularly suitable for use in such agents include the alkyl and/or alkenyl sulfates having 8 to 22 C atoms which carry an alkali-, ammonium- or alkyl- or hydroxyalkyl-substituted ammonium ion as a countercation. Preference is given to the derivatives of the fatty alcohols having in particular 12 to 18 C atoms and their branched-chain analogs, so-called oxo alcohols. The alkyl and alkenyl sulfates can be prepared in a known manner by reaction of the corresponding alcohol component with a conventional sulfating reagent, in particular sulfur trioxide or chlorosulfonic acid, and subsequent neutralization with alkali-, ammonium- or alkyl- or hydroxyalkyl-substituted ammonium bases. The sulfate-type surfactants that can be particularly preferably used include the sulfated alkoxylation products of C₁₂₋₁₈ alcohols, so-called ether sulfates. Such ether sulfates preferably contain from 2 to 30, in particular from 4 to 10, ethylene glycol groups per molecule. Suitable sulfonate-type anionic surfactants include the xx-sulfoesters obtainable by reaction of fatty acid esters with sulfur trioxide and subsequent neutralization, in particular those of fatty acids having 8 to 22 C atoms, preferably 12 to 18 C atoms, and linear alcohols having 1 to 6 C atoms, preferably 1 to 4 C atoms, derivative sulfonation products, as well as the sulfo fatty acids resulting therefrom by formal saponification. The anionic surfactants that may be used also include the salts of alkyl sulfosuccinic acid esters, which are also referred to as alkyl sulfosuccinates or as dialkyl sulfosuccinates and which represent monoesters or diesters of sulfosuccinic acid with alcohols, preferably fatty alcohols, and in particular ethoxylated fatty alcohols. Preferred sulfosuccinates contain C₈-C₁₈ fatty alcohol groups or mixtures thereof. Particularly preferred sulfosuccinates contain an ethoxylated fatty alcohol functional group, which, considered in isolation, represents a nonionic surfactant. Among these, in turn, sulfosuccinates, including fatty alcohol groups that derive from ethoxylated fatty alcohols exhibiting a restricted distribution of homologs, are particularly preferred.

A further embodiment of the agents comprises the presence of alkoxylated C₁₀-C₂₂ alcohols, in which the degree of alkoxylation of the alcohols is below 20, preferably below 10, and which are accessible by reacting corresponding alcohols with alkylene oxides, wherein primary linear or branched-chain alcohols are preferred. Accordingly, the alkoxylates of primary alcohols with linear, in particular decyl, dodecyl, tridecyl, tetradecyl, hexadecyl or octadecyl functional groups and mixtures thereof are usable. The degree of alkoxylation, i.e. the average number of alkoxy groups per alcohol function, can assume integer or fractional numerical values and is preferably in the range from 2 to 10, in particular from 4 to 8. Preferred alkoxy groups are ethoxy, propoxy and butoxy groups, in particular ethoxy groups and mixtures of ethoxy and propoxy groups. Other nonionic surfactants are selected from fatty alkyl polyglycosides, fatty acid polyhydroxyamides and/or ethoxylation and/or propoxylation products of fatty alkylamines, vicinal diols, fatty acid alkyl esters and/or fatty acid amides and mixtures thereof.

Suitable nonionic surfactants include the alkoxylates, in particular the ethoxylates and/or propoxylates, of saturated or mono- to polyunsaturated linear or branched-chain alcohols having 10 to 22 C atoms, preferably 12 to 18 C atoms, in which the degree of alkoxylation of the alcohols is below 20, preferably below 10. They may be produced in a known manner by reacting the corresponding alcohols with the corresponding alkylene oxides. Particularly suitable are the derivatives of fatty alcohols, although their branched-chain isomers, in particular so-called oxo alcohols, can also be used for the preparation of usable alkoxylates. In addition, corresponding alkoxylation products of alkylamines, vicinal diols and carboxylic acid amides, which correspond to said alcohols with respect to the alkyl part, are usable.

Other surface-active ingredients include soaps, with saturated fatty acid soaps, such as the salts of lauric acid, myristic acid, palmitic acid or stearic acid, as well as soaps derived from natural fatty acid mixtures, for example coconut, palm kernel or tallow fatty acids, being suitable. In particular, soap mixtures are preferred which are composed of 50 wt. % to 100 wt. % saturated C₁₂-C₁₈ fatty acid soaps and up to 50 wt. % oleic acid soap. The soaps are preferably in the form of alkali-, ammonium- or alkyl- or hydroxyalkyl-substituted ammonium salts of the fatty acids.

If desired, the agents may also contain betaines and/or cationic surfactants which, if present, are preferably used in amounts of from 0.5 wt. % to 7 wt. %. Among these, esterquats are particularly preferred.

If desired, the agents may contain peroxygen-based bleaching agents, in particular in amounts ranging from 5 wt. % to 70 wt. %, and optionally bleach activators, in particular in amounts ranging from 2 wt. % to 10 wt. %. The suitable bleaching agents are preferably the peroxygen compounds generally used in detergents, such as percarboxylic acids, for example dodecane diperacid or phthaloylaminoperoxicaproic acid, hydrogen peroxide, alkali metal perborate, which may be in the form of tetra- or monohydrate, percarbonate, perpyrophosphate and persilicate, which are generally in the form of alkali salts, in particular sodium salts. Such bleaching agents are present in detergents preferably in amounts of up to 25 wt. %, in particular up to 15 wt. % and particularly preferably from 5 wt. % to 15 wt. %, in each case based on the total agent, in particular percarbonate being used. The optionally present component of the bleach activators comprises the commonly used N- or O-acyl compounds, for example polyacylated alkylenediamines, in particular tetraacetylethylenediamine, acylated glycolurils, in particular tetraacetylglycoluril, N-acylated hydantoins, hydrazides, triazoles, urazoles, diketopiperazines, sulfurylamides and cyanurates, and carboxylic acid anhydrides, in particular phthalic acid anhydride, carboxylic acid esters, in particular sodium isononanoyl phenolsulfonate, and acylated sugar derivatives, in particular pentaacetylglucose, and cationic nitrile derivatives such as trimethylammonium acetonitrile salts. The bleach activators may have been coated with coating substances and/or granulated in a known manner in order to avoid interaction with the percompounds during storage, wherein tetraacetylethylenediamine granulated using carboxymethyl cellulose and having an average particle size of from 0.01 mm to 0.8 mm, granulated 1,5-diacetyl-2,4-dioxohexahydro-1,3,5-triazine, and/or trialkylammonium acetonitrile finished in particulate form is particularly preferred. Such bleach activators are preferably contained in detergents in amounts of up to 8 wt. %, in particular from 2 wt. % to 6 wt. %, in each case based on the total agent.

In a further embodiment, the agent contains water-soluble and/or water-insoluble builders, in particular selected from alkali metal aluminosilicate, crystalline alkali silicate having a modulus above 1, monomeric polycarboxylate, polymeric polycarboxylate and mixtures thereof, in particular in amounts in the range of from 2.5 wt. % to 60 wt. %.

The agent preferably contains 20 wt. % to 55 wt. % water-soluble and/or water-insoluble, organic and/or inorganic builders. The water-soluble organic builders include in particular those from the class of the polycarboxylic acids, in particular citric acid and saccharic acids, and the polymeric (poly) carboxylic acids, in particular the polycarboxylates obtainable by oxidation of polysaccharides, polymeric acrylic acids, methacrylic acids, maleic acids and mixed polymers thereof, which may also contain, in the polymer, small portions of polymerizable substances, without a carboxylic acid functionality. The relative molecular mass of the homopolymers of unsaturated carboxylic acids is generally between 5,000 g/mol and 200,000 g/mol, that of the copolymers is between 2,000 g/mol and 200,000 g/mol, preferably 50,000 g/mol to 120,000 g/mol, based on free acid. A particularly preferred acrylic acid-maleic acid copolymer has a relative molecular mass of from 50,000 g/mol to 100,000 g/mol. Suitable, albeit less preferred compounds of this class are copolymers of acrylic acid or methacrylic acid with vinyl ethers, such as vinyl methyl ethers, vinyl esters, ethylene, propylene, and styrene, in which the proportion of the acid is at least 50 wt. %. It is also possible to utilize, as water-soluble organic builders, terpolymers which contain two carboxylic acids and/or the salts thereof as monomers and vinyl alcohol and/or a vinyl alcohol derivative or a carbohydrate as the third monomer. The first acidic monomer or the salt thereof is derived from a monoethylenically unsaturated C₃-C₈ carboxylic acid and preferably from a C₃-C₄ monocarboxylic acid, in particular from (meth)acrylic acid. The second acidic monomer or salt thereof may be a derivative of a C₄-C₈ dicarboxylic acid, with maleic acid being particularly preferred. In this case, the third monomeric unit is formed from vinyl alcohol and/or preferably an esterified vinyl alcohol. In particular, vinyl alcohol derivatives are preferred, which are an ester of short-chain carboxylic acids, for example C₁-C₄ carboxylic acids, with vinyl alcohol. Preferred terpolymers contain 60 wt. % to 95 wt. %, in particular 70 wt. % to 90 wt. %, of (meth)acrylic acid and/or (meth)acrylate, particularly preferably acrylic acid and/or acrylate, and maleic acid and/or maleinate, and 5 wt. % to 40 wt. %, preferably 10 wt. % to 30 wt. %, of vinyl alcohol and/or vinyl acetate. Terpolymers in which the weight ratio of (meth)acrylic acid and/or (meth)acrylate to maleic acid and/or maleate is between 1:1 and 4:1, preferably between 2:1 and 3:1 and in particular 2:1 and 2.5:1, are very particularly preferred. Both the amounts and the weight ratios are based on the acids. The second acidic monomer or the salt thereof can also be a derivative of an allyl sulfonic acid which is substituted in the 2 position with an alkyl functional group, preferably with a C₁-C₄ alkyl functional group, or an aromatic functional group which is preferably derived from benzene or benzene derivatives. Preferred terpolymers contain 40 wt. % to 60 wt. %, in particular 45 to 55 wt. %, of (meth)acrylic acid and/or (meth)acrylate, particularly preferably acrylic acid and/or acrylate, 10 wt. % to 30 wt. %, preferably 15 wt. % to 25 wt. % of methallyl sulfonic acid and/or methallyl sulfonate and, as a third monomer, 15 wt. % to 40 wt. %, preferably 20 wt. % to 40 wt. % of a carbohydrate. This carbohydrate may be, for example, a mono-, di-, oligo- or polysaccharide, mono-, di- or oligosaccharides being preferred, sucrose particularly being preferred. The use of the third monomer presumably incorporates predetermined breaking points into the polymer which are responsible for the good biodegradability of the polymer. These terpolymers generally have a relative molecular mass between 1,000 g/mol and 200,000 g/mol, preferably between 2,000 g/mol and 50,000 g/mol, and in particular between 3,000 g/mol and 10,000 g/mol. They may be used in the form of aqueous solutions, preferably in the form of 30 to 50 wt. % aqueous solutions, in particular for the preparation of liquid agents. All of the polycarboxylic acids mentioned are generally used in the form of their water-soluble salts, in particular their alkali salts.

Organic builders of this kind are preferably contained in amounts of up to 40 wt. %, in particular up to 25 wt. %, and particularly preferably from 1 wt. % to 5 wt. %. Amounts close to the stated upper limit are preferably used in pasty or liquid, in particular water-containing, agents.

In particular crystalline or amorphous alkali aluminosilicates are used as water-insoluble, water-dispersible inorganic builder materials in amounts of up to 50 wt. %, preferably no greater than 40 wt. %, and in liquid agents in particular in amounts of from 1 wt. % to 5 wt. %. Among these, the crystalline aluminosilicates of detergent grade, in particular zeolite NaA and optionally NaX, are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm. The calcium binding capacity of said aluminosilicates, which can be determined according to the specifications in German patent DE 24 12 837, is generally in the range of from 100 to 200 mg CaO per gram. Suitable substitutes or partial substitutes for the aluminosilicate mentioned are crystalline alkali silicates, which may be present alone or in a mixture with amorphous silicates. The alkali silicates that can be used in the agents as builders preferably have a molar ratio of alkali oxide to SiO₂ of less than 0.95, in particular from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. Such amorphous alkali silicates are commercially available, for example, under the name Portil®. Those with a molar ratio Na₂O:SiO₂ of 1:1.9 to 1:2.8 are preferably added as a solid during production and not in the form of a solution. Crystalline phyllosilicates of the general formula Na₂SiₓO₂ₓ₊₁·yH₂O, where x, referred to as the modulus, is a number from 1.9 to 4, y is a number from 0 to 20, and preferred values for x are 2, 3 or 4, are preferably used as crystalline silicates, which may be present on their own or in a mixture with amorphous silicates. Crystalline phyllosilicates covered by this general formula are described, for example, in European patent application EP 0 164 514. Preferred crystalline phyllosilicates are those in which x assumes the values 2 or 3 in the mentioned general formula. In particular, both β- and 8-sodium disilicates (Na₂Si₂O₅·yH₂O) are preferred. Practically water-free crystalline alkali silicates which have the above general formula, in which x is a number from 1.9 to 2.1, and which are prepared from amorphous alkali silicates may also be used in agents which contain an active ingredient combination to be used according to the invention. In another preferred embodiment of agents according to the invention, a crystalline sodium phyllosilicate having a modulus of 2 to 3 is used, such as can be produced from sand and soda ash. Crystalline sodium silicates having a modulus in the range of from 1.9 to 3.5 are used in a further preferred embodiment of detergents which contain an active ingredient combination used according to the invention. Their content of alkali silicates is preferably from 1 wt. % to 50 wt. % and in particular 5 wt. % to 35 wt. %, based on water-free active substance. If alkali metal aluminosilicate, in particular zeolite, is present as an additional builder, the content of alkali silicate is preferably 1 wt. % to 15 wt. % and in particular 2 wt. % to 8 wt. %, based on water-free active substance. The weight ratio of aluminosilicate to silicate, in each case based on water-free active substances, is then preferably 4:1 to 10:1. In agents containing amorphous and crystalline alkali silicates, the weight ratio of amorphous alkali silicate to crystalline alkali silicate is preferably from 1:2 to 2:1 and in particular from 1:1 to 2:1.

In addition to the aforementioned inorganic builder, other water-soluble or water-insoluble inorganic substances may be contained in the agents which contain an active ingredient combination to be used according to the invention, are used together therewith or are used in the method according to the invention. Suitable in this context are the alkali metal carbonates, alkali metal bicarbonates and alkali metal sulfates as well as mixtures thereof. Such additional inorganic material may be present in amounts of up to 70 wt. %.

In a preferred embodiment of the invention, an agent according to the invention comprises a water-soluble builder block. The use of the term “builder block” is intended to express the fact that the agents do not contain any builders other than those that are water-soluble, i.e. all the builders contained in the agent are combined in the “block” thus characterized, with the exception of any amounts of substances that may customarily be present as impurities or stabilizing additives in small quantities in the other ingredients of the agent. The term “water-soluble” is to be understood as meaning that the builder block dissolves without residue at the concentration resulting from the amount of agent used that contains said builder block under normal conditions. Preferably, at least 15 wt. % and up to 55 wt. %, in particular 25 wt. % to 50 wt. %, of water-soluble builder block are contained in the agents according to the invention. This is preferably composed of the components
a) 5 wt. % to 35 wt. % citric acid, alkali citrate and/or alkali carbonate, which may also be at least partially replaced by alkali hydrogen carbonate,
b) up to 10 wt. % alkali silicate with a modulus in the range from 1.8 to 2.5,
c) up to 2 wt. % phosphonic acid and/or alkali phosphonate,
d) up to 50 wt. % alkali phosphate, and
e) up to 10 wt. % polymeric polycarboxylate,
wherein the quantities are stated in relation to the total detergent or cleaning agent.

In a preferred embodiment of agents according to the invention, the water-soluble builder block contains at least 2 of components b), c), d) and e) in amounts greater than 0 wt. %.

With regard to component a), a preferred embodiment of agents according to the invention contains 15 wt. % to 25 wt. % of alkali carbonate, which can be at least partially replaced by alkali hydrogen carbonate, and up to 5 wt. %, in particular 0.5 wt. % to 2.5 wt. %, of citric acid and/or alkali citrate. An alternative embodiment of agents according to the invention contains, as component a), 5 wt. % to 25 wt. %, in particular 5 wt. % to 15 wt. %, citric acid and/or alkali citrate and up to 5 wt. %, in particular 1 wt. % to 5 wt. %, alkali carbonate, which can be at least partially replaced by alkali hydrogen carbonate. If both alkali carbonate and alkali hydrogen carbonate are present, component a) preferably comprises alkali carbonate and alkali hydrogen carbonate in a weight ratio of 10:1 to 1:1.

With regard to component b), a preferred embodiment of agents according to the invention contains 1 wt. % to 5 wt. % of alkali silicate with a modulus in the range of from 1.8 to 2.5.

With regard to component c), a preferred embodiment of agents according to the invention contains 0.05 wt. % to 1 wt. % of phosphonic acid and/or alkali phosphonate. Phosphonic acids are also understood as meaning optionally substituted alkyl phosphonic acids which can also have a plurality of phosphonic acid groups (so-called polyphosphonic acids). They are preferably selected from the hydroxy and/or aminoalkylphosphonic acids and/or their alkali salts, such as dimethylaminomethanediphosphonic acid, 3-aminopropane-1-hydroxy-1,1-diphosphonic acid, 1-amino-1-phenylmethanediphosphonic acid, 1-hydroxyethane-1,1-diphosphonic acid, aminotris(methylenephosphonic acid), N,N,N′,N′-ethylenediaminetetrakis(methylenephosphonic acid), and acylated derivatives of the phosphorous acid, which can also be used in any mixtures.

With regard to component d), a preferred embodiment of agents according to the invention contains 15 wt. % to 35 wt. % of alkali phosphate, in particular trisodium polyphosphate.

With regard to component e), a preferred embodiment of agents according to the invention contains 1.5 wt. % to 5 wt. % of polymeric polycarboxylate, in particular selected from the polymerization or copolymerization products of acrylic acid, methacrylic acid and/or maleic acid. Among these, the homopolymers of acrylic acid and, in turn among these, those with an average molar mass in the range of 5,000 g/mol to 15,000 g/mol (PA standard) are particularly preferred.

In addition, the agents may contain other components that are conventional in detergents or cleaning agents. These optional ingredients include, in particular, enzymes, enzyme stabilizers, complexing agents for heavy metals, for example aminopolycarboxylic acids, aminohydroxypolycarboxylic acids, polyphosphonic acids and/or aminopolyphosphonic acids, suds suppressors, for example organopolysiloxanes or paraffins, solvents and optical brighteners, for example stilbene disulfonic acid derivatives. Preferably, agents which contain an active ingredient combination used according to the invention contain up to 1 wt. %, in particular 0.01 wt. % to 0.5 wt. %, optical brighteners, in particular compounds from the class of the substituted 4,4′-bis(2,4,6-triamino-s-triazinyl)-stilbene-2,2′-disulfonic acids, up to 5 wt. %, in particular from 0.1 wt. % to 2 wt. %, complexing agent for heavy metals, in particular aminoalkylenephosphonic acids and salts thereof, and up to 2 wt. %, in particular from 0.1 wt. % to 1 wt. %, suds suppressors, the percentages by weight given each relating to the total agent.

Solvents which can be used in particular for liquid agents are, in addition to water, preferably non-aqueous solvents that are water-miscible. These include the lower alcohols, for example ethanol, propanol, isopropanol, and the isomeric butanols, glycerol, lower glycols, such as ethylene and propylene glycol, and the ethers derivable from said classes of compounds. In such liquid agents, the active ingredients used according to the invention are usually dissolved or in suspended form.

Optionally present enzymes are preferably selected from the group comprising protease, amylase, lipase, cellulase, hemicellulase, oxidase, peroxidase, pectinase and mixtures thereof. First and foremost, proteases derived from microorganisms, such as bacteria or fungi, come into question. Protease can be obtained in a known manner by fermentation processes from suitable microorganisms. Proteases are commercially available, for example, under the names BLAP®, Savinase®, Esperase®, Maxatase®, Optimase®, Alcalase®, Durazym® or Maxapem®. The lipase which can be used can be obtained, for example, from Humicola lanuginosa, from Bacillus species, from Pseudomonas species, from Fusarium species, from Rhizopus species or from Aspergillus species. Suitable lipases are commercially available, for example, under the names Lipolase®, Lipozym®, Lipomax®, Lipex®, Amano®-Lipase, Toyo-Jozo®-Lipase, Meito®-Lipase and Diosynth®-Lipase. Suitable amylases are commercially available, for example, under the names Maxamyl®, Termamyl®, Duramyl® and Purafect® OxAm. The cellulase which can be used may be an enzyme which can be obtained from bacteria or fungi and has an optimum pH preferably in the slightly acidic to slightly alkaline range of from 6 to 9.5. Such cellulases are commercially available under the names Celluzyme®, Carezyme® and Ecostone®. Suitable pectinases are available, for example, under the names Gamanase®, Pektinex AR®, X-Pect® or Pectaway® from Novozymes, under the names Rohapect UF®, Rohapect TPL®, Rohapect PTE100®, Rohapect MPER, Rohapect MA plus HC, Rohapect DA12L®, Rohapect 10L®, Rohapect B1L® from AB Enzymes, and under the name Pyrolase® from Diversa Corp., San Diego, CA, USA.

Conventional enzyme stabilizers that are optionally present in particular in liquid agents include amino alcohols, for example mono-, di-, triethanol- and -propanolamine and mixtures thereof, lower carboxylic acids, boric acid, alkali borates, boric acid-carboxylic acid combinations, boric acid esters, boronic acid derivatives, calcium salts, for example, a Ca-formic acid combination, magnesium salts, and/or sulfur-containing reducing agents.

Suitable suds suppressors include long-chain soaps, in particular behenic soap, fatty acid amides, paraffins, waxes, microcrystalline waxes, organopolysiloxanes and mixtures thereof, which moreover can contain microfine, optionally silanated or otherwise hydrophobized silicic acid. For use in particulate agents, such suds suppressors are preferably bound to granular, water-soluble carrier substances.

The known polyester-active polymers which allow the removal of dirt and which can be used in addition to the active ingredient combination essential to the invention include copolyesters of dicarboxylic acids, for example adipic acid, phthalic acid or terephthalic acid, diols, for example ethylene glycol or propylene glycol, and polydiols, for example polyethylene glycol or polypropylene glycol. Preferred polyesters which allow the removal of dirt include those compounds which are formally accessible by esterification of two monomeric moieties, the first monomer being a dicarboxylic acid HOOC-Ph-COOH and the second monomer being a diol HO—(CHR¹¹—)ₐOH, which can also be present as polymeric diol H—(O—(CHR₁₁—)ₐ)_{b}OH. Here, Ph denotes an o-, m- or p-phenylene functional group which can carry 1 to 4 substituents selected from alkyl functional groups having 1 to 22 C atoms, sulfonic acid groups, carboxyl groups, and mixtures thereof, R¹¹ denotes hydrogen, an alkyl functional group having 1 to 22 C atoms and mixtures thereof, a denotes a number from 2 to 6 and b denotes a number from 1 to 300. Preferably, in the polyesters obtainable from these, both monomer diol units —O—(CHR₁₁—)ₐO— and also polymeric diol units-(O—(CHR₁₁—)ₐ)_{b}O— are present. The molar ratio of monomer diol units to polymer diol units is preferably from 100:1 to 1:100, in particular 10:1 to 1:10. In the polymer diol units, the degree of polymerization b is preferably in the range of from 4 to 200, in particular from 12 to 140. The molecular weight or the average molecular weight or the maximum molecular weight distribution of preferred polyesters which allow the removal of dirt is in the range of from 250 g/mol to 100,000 g/mol, in particular from 500 g/mol to 50,000 g/mol. The acid underlying the functional group Ph is preferably selected from terephthalic acid, isophthalic acid, phthalic acid, trimellitic acid, mellitic acid, the isomers of sulfophthalic acid, sulfoisophthalic acid and sulfoterephthalic acid and mixtures thereof. If their acid groups are not part of the ester bonds in the polymer, they are preferably in salt form, in particular as alkali or ammonium salt. Among these, the sodium and potassium salts are particularly preferred. If desired, small proportions, in particular no more than 10 mol. % based on the proportion of Ph having the meaning given above, of other acids having at least two carboxyl groups may be present in the polyester which allows the removal of dirt instead of the monomer HOOC-Ph-COOH. These include, for example, alkylene and alkenylene dicarboxylic acids such as malonic acid, succinic acid, fumaric acid, maleic acid, glutaric acid, adipic acid, pimelic acid, suberic acid, azelaic acid and sebacic acid. Preferred diols HO—(CHR₁₁—)ₐOH include those in which R¹¹ is hydrogen and a is a number from 2 to 6, and those in which a has the value 2 and R¹¹ is selected from hydrogen and the alkyl functional groups having 1 to 10, in particular 1 to 3, C atoms. Among the last-mentioned diols, those of formula HO—CH₂—CHR₁₁—OH in which R¹¹ has the above-mentioned meaning are particularly preferred. Examples of diol components are ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol and neopentyl glycol. Particularly preferred of the polymeric diols is polyethylene glycol having an average molar mass in the range of from 1,000 g/mol to 6,000 g/mol. If desired, the polyesters may also be end-capped, with alkyl groups having 1 to 22 C atoms and esters of monocarboxylic acids being suitable as end groups. The end groups bonded via ester bonds can be based on alkyl, alkenyl and aryl monocarboxylic acids having 5 to 32 C atoms, in particular 5 to 18 C atoms. These include valeric acid, caproic acid, enanthic acid, caprylic acid, pelargonic acid, capric acid, undecanoic acid, undecenoic acid, lauric acid, lauroleinic acid, tridecanoic acid, myristic acid, myristoleic acid, pentadecanoic acid, palmitic acid, stearic acid, petroselic acid, petroselaidic acid, oleic acid, linoleic acid, linolaidic acid, linolenic acid, eleostearic acid, arachidic acid, gadoleic acid, arachidonic acid, behenic acid, erucic acid, brassic acid, clupanodonic acid, lignoceric acid, cerotic acid, melissic acid, benzoic acid, which may carry 1 to 5 substituents having a total of up to 25 C atoms, in particular 1 to 12 C atoms, for example tert-butylbenzoic acid. The end groups can also be based on hydroxymonocarboxylic acids having 5 to 22 C atoms, which include, for example, hydroxyvaleric acid, hydroxycaproic acid, ricinoleic acid, the hydrogenation product of which includes hydroxystearic acid and o-, m- and p-hydroxybenzoic acid. The hydroxymonocarboxylic acids may in turn be linked to one another via their hydroxyl group and their carboxyl group and thus be present several times in an end group. Preferably, the number of hydroxymonocarboxylic acid units per end group, i.e., their degree of oligomerization, is in the range of from 1 to 50, in particular from 1 to 10. In a preferred embodiment of the invention, polymers of ethylene terephthalate and polyethylene oxide terephthalate, in which the polyethylene glycol units have molecular weights of from 750 g/mol to 5,000 g/mol and the molar ratio of ethylene terephthalate to polyethylene oxide terephthalate is 50:50 to 90:10, are used together with an active ingredient combination that is essential to the invention. The polymers which allow the removal of dirt are preferably water-soluble, the term “water-soluble” being understood to mean having a solubility of at least 0.01 g, preferably at least 0.1 g, of the polymer per liter of water at room temperature and pH 8. However, polymers that are preferably used have a solubility of at least 1 g per liter, in particular at least 10 g per liter, under these conditions.

The preparation of solid agents according to the invention presents no difficulties and can be carried out in a known manner, for example by spray-drying or granulation, wherein enzymes and possibly other thermally sensitive ingredients, such as bleaching agents, are optionally added separately later. For the production of agents according to the invention having an increased bulk weight, in particular in the range of from 650 g/l to 950 g/l, a method having an extrusion step is preferred.

To prepare agents according to the invention in tablet form, which can be single-phase or multiphase, single-color or multi-color, and in particular can be composed of one layer or of a plurality of layers, in particular of two layers, all components—optionally a layer of each—are preferably mixed with one another in a mixer, and the mixture is compressed using conventional tablet presses, such as eccentric presses or rotary tablet presses, using pressures in the range of from approximately 50 to 100 kN, preferably of 60 to 70 kN. In the case of multilayer tablets in particular, it can be advantageous if at least one layer is pre-pressed. This is preferably carried out at pressures of between 5 and 20 kN, in particular at 10 to 15 kN. This readily yields break-resistant tablets that nonetheless dissolve sufficiently quickly under usage conditions, normally with breaking and flexural strengths of 100 to 200 N, but preferably above 150 N. A tablet thus produced preferably has a weight of 10 g to 50 g, and in particular of 15 g to 40 g. The tablets can have any physical shape, and they can be round, oval, or angular, wherein intermediate shapes are also possible. Corners and edges are advantageously rounded. Round tablets preferably have a diameter of 30 mm to 40 mm. In particular, the size of angular or cuboid tablets, which are predominantly introduced via the dosing device, for example of the dishwasher, is dependent on the geometry and the volume of this dosing device. By way of example, preferred embodiments have a base area of (20 to 30 mm)×(34 to 40 mm), and in particular of 26×36 mm or of 24×38 mm.

Liquid or pasty agents according to the invention in the form of solutions containing conventional solvents, in particular water, are usually prepared by simply mixing the ingredients, which can be added to an automatic mixer in bulk or as a solution.

In a further preferred embodiment, an agent according to the invention is ready-portioned as a single dose in a chamber formed from water-soluble material; the agent then preferably contains less than 15 wt. %, in particular in the range of 1 wt. % to 12 wt. %, water. A portion is an independent dosing unit having at least one chamber in which the product to be dosed is contained. A chamber is a space delimited by walls (e.g. by a film), which space can also exist without the product to be dosed (optionally by changing its shape). A surface coating or a layer of a surface coating is therefore not a wall according to the present invention.

The walls of the chamber are made of a water-soluble material. The water solubility of the material can be determined by means of a square film of said material (film: 22×22 mm with a thickness of 76 μm) fixed in a square frame (edge length on the inside: 20 mm) according to the following measurement protocol. Said framed film is submerged into 800 ml of distilled water, temperature-controlled to 20° C., in a 1 liter glass beaker with a circular base (Schott, Mainz, 1000 ml glass beaker, low shape), so that the surface of the tensioned film is arranged at a right angle to the base of the beaker, the upper edge of the frame is 1 cm below the water surface, and the lower edge of the frame is oriented parallel to the base of the glass beaker such that the lower edge of the frame extends along the radius of the base of the glass beaker and the center of the lower edge of the frame is arranged above the center of the radius of the glass beaker bottom. The material dissolves within 600 seconds when stirred (stirring speed magnet stirrer 300 rpm, stirring rod: 5 cm long), such that no solid particles at all can be seen with the naked eye.

The walls of the chambers and therefore the water-soluble wrappings of the detergents according to the invention are preferably formed by a water-soluble film material. Water-soluble packages of this kind can be produced either by means of methods of vertical form fill sealing or by means of thermoforming methods.

The thermoforming method generally includes forming a first layer from a water-soluble film material in order to produce bulges for receiving a composition, pouring the composition into the bulges, covering the bulges filled with the composition with a second layer made of a water-soluble film material, and sealing the first and second layers to one another at least around the bulges.

The water-soluble film material is preferably selected from polymers or polymer mixtures. The wrapping may be made up of one or of two or more layers of water-soluble film material. The water-soluble film materials of the first layer and of the additional layers, if present, may be the same or different.

It is preferable for the water-soluble wrapping to contain polyvinyl alcohol or a polyvinyl alcohol copolymer; particularly preferably, it consists of polyvinyl alcohol or polyvinyl alcohol copolymer.

Water-soluble films for preparing the water-soluble wrapping are preferably based on a polyvinyl alcohol or a polyvinyl alcohol copolymer of which the molecular weight is in the range of from 10,000 to 1,000,000 gmol⁻¹, preferably from 20,000 to 500,000 gmol⁻¹, particularly preferably from 30,000 to 100,000 gmol⁻¹, and in particular from 40,000 to 80,000 gmol⁻¹.

Polyvinyl alcohol is usually prepared by hydrolysis of polyvinyl acetate, since the direct synthesis route is not possible. The same applies to polyvinyl alcohol copolymers, which are prepared accordingly from polyvinyl acetate copolymers. It is preferable for at least one layer of the water-soluble wrapping to include a polyvinyl alcohol of which the degree of hydrolysis is 70 to 100 mol. %, preferably 80 to 90 mol. %, particularly preferably 81 to 89 mol. %, and in particular 82 to 88 mol. %.

Polymers selected from the group comprising acrylic acid-containing polymers, polyacrylamides, oxazoline polymers, polystyrene sulfonates, polyurethanes, polyesters, polyethers, polylactic acid, and/or mixtures of the above polymers may additionally be added to a film material suitable for producing the water-soluble wrapping. It is also possible to copolymerize such monomers on which the polymers are based, individually or in mixtures of two or more, with vinyl acetate.

Preferred polyvinyl alcohol copolymers comprise, in addition to vinyl alcohol, an ethylenically unsaturated carboxylic acid, or the salt or ester thereof. Polyvinyl alcohol copolymers of this kind particularly preferably contain acrylic acid, methacrylic acid, acrylic acid ester, methacrylic acid ester or mixtures thereof, in addition to vinyl alcohol; of the esters, C₁₋₄ alkyl esters or C₁₋₄ hydroxyalkyl esters are preferred. Polyvinyl alcohol copolymers which include, in addition to vinyl alcohol, ethylenically unsaturated dicarboxylic acids as further monomers are also preferred. Suitable dicarboxylic acids are, for example, itaconic acid, maleic acid, fumaric acid, and mixtures thereof, itaconic acid being particularly preferred.

Suitable water-soluble films for use in the wrappings of the water-soluble packaging according to the invention are films which are sold by MonoSol LLC, for example under the names M8630, C8400 or M8900. Other suitable films include films with the name Solublon® PT, Solublon® GA, Solublon® KC or Solublon® KL by Aicello Chemical Europe GmbH or the VF—HP films by Kuraray.

The detergent portion comprising the detergent and the water-soluble wrapping may comprise one or more chambers. The water-soluble wrappings having a chamber can have a substantially dimensionally stable spherical, rotationally ellipsoidal, cubic, cuboidal or pillow-shaped design with a circular, elliptical, square or rectangular basic shape. The agent may be contained in one or more chambers, if present, of the water-soluble wrapping.

In a preferred embodiment, the water-soluble wrapping has two chambers. In this embodiment, the two chambers may each contain a solid partial composition or a liquid partial composition, or the first chamber contains a liquid partial composition and the second chamber a solid partial composition.

The proportions of the agents contained in the different chambers of a water-soluble wrapping having two or more chambers may have the same composition. Preferably, however, the agents in a water-soluble wrapping having at least two chambers have partial compositions which differ by at least one ingredient and/or by the content of at least one ingredient. Preferably, a partial composition of such agents according to the invention comprises an enzyme and/or a bleach activator and a separate further partial composition comprises peroxidic bleaching agent, the first partial composition thus in particular not comprising peroxidic bleaching agent and the second partial composition in particular not comprising an enzyme or a bleach activator.

Packaging portions in a water-soluble wrapping allows the user, for one application, to place one or, if desired, more, preferably one, of the portions into a washing machine, in particular into the detergent drawer of a washing machine, or into a container for carrying out a manual washing method. Portion packs of this kind fulfil the consumer's desire for simplified dosage. After the addition of water, the wrapping material dissolves such that the ingredients can be released and take effect in the liquor. Preferably, a water-solubly wrapped portion weighs 10 g to 35 g, in particular 12 g to 28 g, and particularly preferably 12 g to 15 g, where 0.3 g to 2.5 g, in particular 0.7 g to 1.2 g, of the content of the water-soluble wrapping included in the stated weight is omitted.

### EXAMPLES

A compound according to general formula (I) was used, the functional groups R of which consisted of 72% H and 28% of a 55:45 mixture of octanoic acid esters and decanoic acid esters (M1). For comparison, a compound (V1) otherwise corresponding to general formula (I) was used, the functional groups R of which consisted of 100% H. From these, the detergents W2 and W3 indicated in the table below in their composition (wt. %) were produced. For comparison, a compound W1 that was free of both was also tested.

TABLE 1
Composition of the detergents [wt. %]

| Ingredient / agent | W1 | W2 | W3 |
|---|---|---|---|
| C₁₂₋₁₈ fatty alcohol ethoxylate, 7 EO | 24.90 | 24.90 | 24.90 |
| C₁₀₋₁₃ alkylbenzenesulfonic acid | 25.05 | 25.05 | 25.05 |
| C₁₂₋₁₈ fatty acid | 8.56 | 8.56 | 8.56 |
| Monoethanolamine | 7.04 | 7.04 | 7.04 |
| 1,2-propanediol | 4.61 | 4.61 | 4.61 |
| Glycerol | 11.04 | 11.04 | 11.04 |
| Na-diethylenetriamine penta(methylene | 1.88 | 1.88 | 1.88 |
| phosphonate) | | | |
| M1 | — | — | 5.80 |
| V1 | — | 5.80 | — |

| | |
|---|---|
| Water | to make up to 100 |

These were used to wash cotton textile items that had been soiled with the standardized soiling specified in Table 2 below for 150 minutes at 20° C. with a main wash cycle duration of 90 minutes, and the increase in washing power of agents W2 and W3 compared to the washing power of agent W1 (brightness differences ΔY) was determined:

TABLE 2
Percent increase in washing power [%]

| Soiling / agent | W2 | W3 |
|---|---|---|
| Butterfat | 0.3 | 4.5 |
| Lipstick | 1.5 | 6.4 |
| Mineral oil/soot | 0.3 | 9.0 |

It can be seen that agent W3 according to the invention has a particularly high increase in washing power.

1. A detergent comprising a compound having the formula (I): 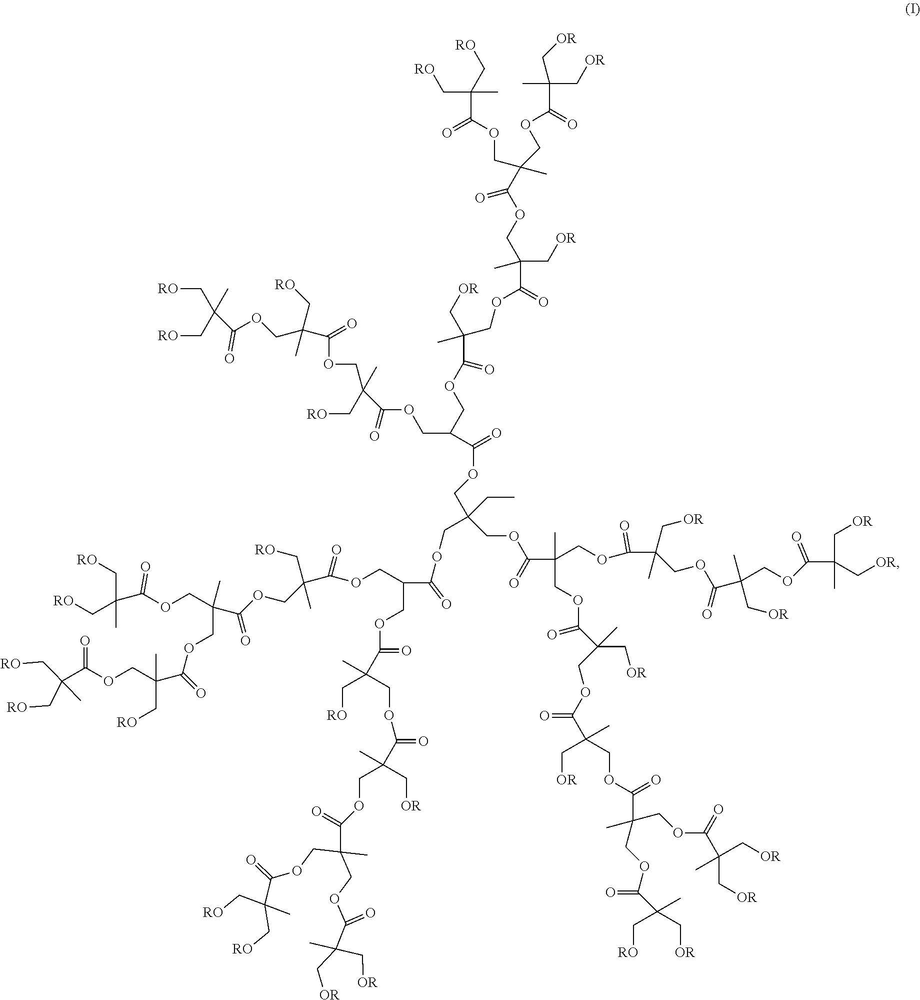
wherein:
each R is —H or —O—C(═O)—R′,
R′ is selected from the group consisting of alkyl groups having 6 to 10 C atoms and mixtures thereof, and
at least 5% of the number of functional groups R are those which are not H.

2. The detergent of claim 1, wherein the compound is in an amount of from 1 wt. % to 6 wt. %, based on the total weight of the detergent.

3. The detergent of claim 1, wherein the detergent is a liquid.

4. The detergent of claim 3, wherein the detergent is ready-portioned as a single dose in a chamber formed from a water-soluble material.

5. The detergent of claim 1, further comprising a synthetic anionic surfactant in an amount of from 10 wt. % to 40 wt. %, based on the total weight of the detergent.

6. A method for increasing the washing power of a detergent against grease-based or oil-based textile soiling using a compound, the method comprising applying the detergent and the compound to one or more textiles, the compound having the formula (I): 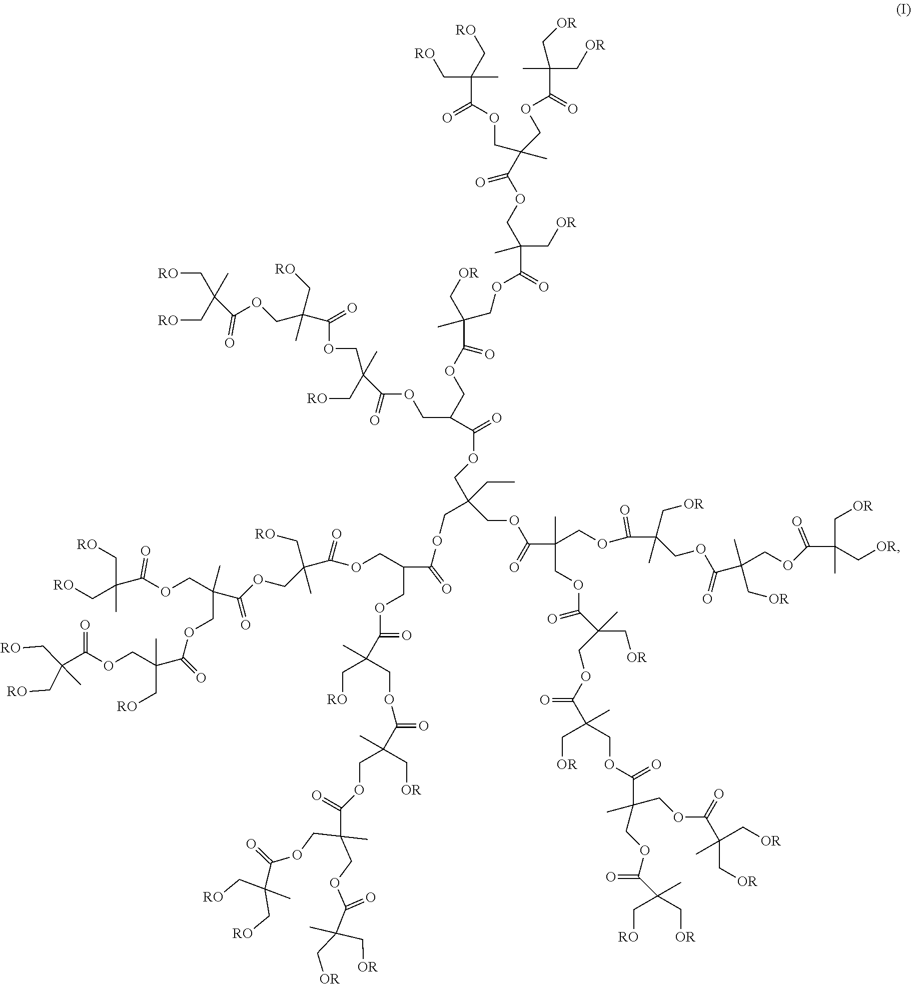
wherein:
each R is —H or —O—C(═O)—R′,
R′ is selected from the group consisting of alkyl groups having 6 to 10 C atoms and mixtures thereof, and
at least 5% of the number of functional groups R are those which are not H.

7. A method for removing grease-based or oil-based soiling from textiles, the method comprising contacting the textiles with water and a compound having the formula (I): 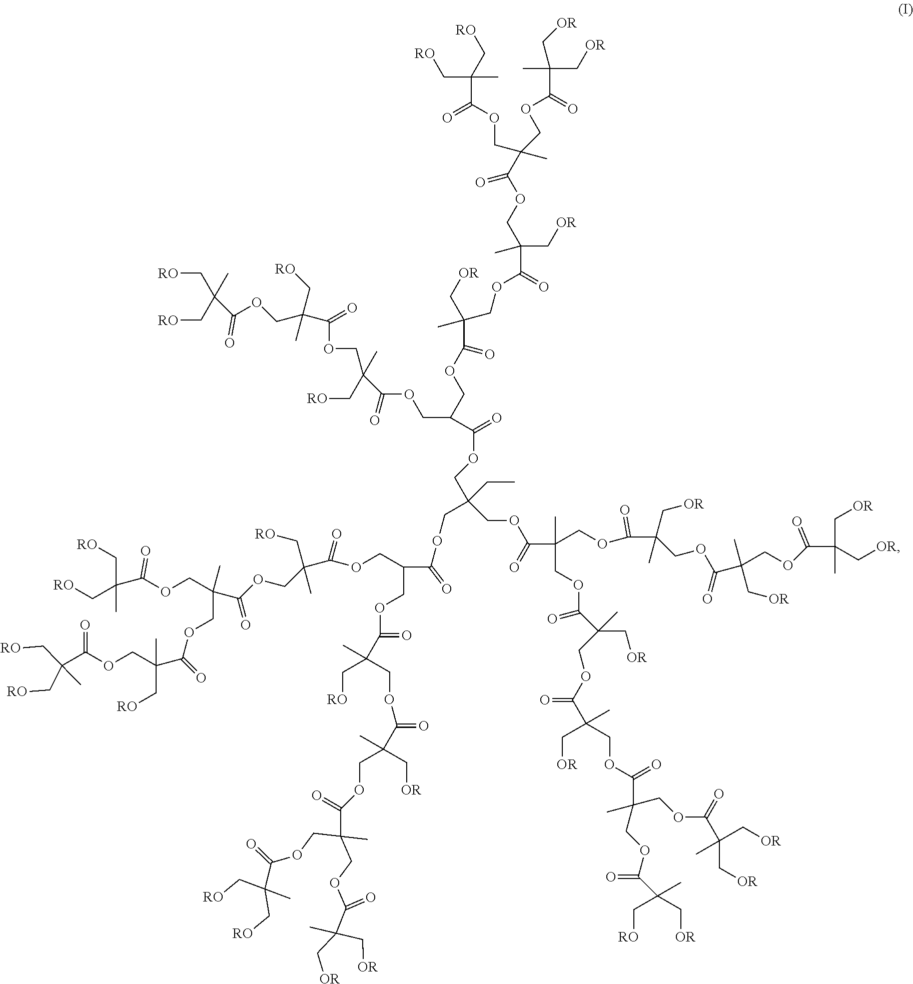
wherein:
each R is —H or —O—C(═O)—R′,
R′ is selected from the group consisting of alkyl groups having 6 to 10 C atoms and mixtures thereof, and
at least 5% of the number of functional groups R are those which are not H.

8. The method of claim 7, wherein the textiles are contacted with the water and the compound at a temperature of from room temperature to 60° C.

9. The detergent of claim 1, wherein 20% to 35% of the number of functional groups R are those which are not H.

10. The detergent of claim 1, wherein 35% to 65% of the number of functional groups R′ in the functional groups R which are not H are those having a C₇ alkyl group.

11. The detergent of claim 2, wherein the compound is in an amount of from 2 wt. % to 4 wt. %, based on the total weight of the detergent.

12. The detergent of claim 4, further comprising water in an amount of up to 15 wt. %, based on the total weight of the detergent.

13. The detergent of claim 1, further comprising a nonionic surfactant in an amount of from 20 wt. % to 40 wt. %, based on the total weight of the detergent.

14. The detergent of claim 1, further comprising a soap in an amount of from 5 wt. % to 20 wt. %, based on the total weight of the detergent.

15. The detergent of claim 1, further comprising a synthetic anionic surfactant, a nonionic surfactant, and a soap.

16. The method of claim 8, wherein the textiles are contacted with the water and the compound at a temperature of from 20° C. to 40° C.

17. The detergent of claim 9, wherein 25% to 30% of the number of functional groups R are those which are not H.

18. The detergent of claim 10, wherein 40% to 60% of the number of functional groups R′ in the functional groups R which are not H are those having the C₇ alkyl group.

19. The detergent of claim 1, wherein 35% to 65% of the number of functional groups R′ in the functional groups R which are not H are those having a C₉ alkyl group.

20. The detergent of claim 19, wherein 40% to 60% of the number of functional groups R′ in the functional groups R which are not H are those having the C₉ alkyl group.
